(19) United States
(12) Patent Application Publication
(10) Pub. No.: US 2026/0178092 A1
(43) Pub. Date: Jun. 25, 2026

(54) ENERGY EFFICIENT SCHEDULING FOR COMPUTING SYSTEMS AND METHOD THEREFOR

(71) Applicant: The Research Foundation for The State University of New York, Binghamton, NY (US)

(72) Inventor: Kanad Ghose, Vestal, NY (US)

(21) Appl. No.: 19/435,391

(22) Filed: Dec. 29, 2025

Related U.S. Application Data
(63) Continuation of application No. 18/425,536, filed on Jan. 29, 2024, now Pat. No. 12,510,943.
Application No. 18/425,536 is a division of application No. 17/473,107, filed on Sep. 13, 2021, now Pat. No. 11,886,914.
Application No. 17/473,107 is a continuation of application No. 15/490,525, filed on Apr. 18, 2017, now abandoned.
Application No. 15/490,525 is a continuation of application No. 12/841,160, filed on Jul. 21, 2010, now abandoned.
(60) Provisional application No. 61/227,361, filed on Jul. 21, 2009.

(51) Int. Cl. G06F 1/20; A61K 36/185; B03B 1/00; B03B 5/02; B03B 5/58; B03B 11/00; G05D 23/19; G06F 1/3203; G06F 1/3206; G06F 1/3209; G06F 1/3228; G06F 9/455; G06F 9/48; G06F 9/50; G06F 13/40; G06K 19/07; G06K 19/077; H04L 69/329; H05K 7/20

(52) U.S. Cl. CPC G06F 1/206; A61K 36/185; B03B 1/00; B03B 5/02; B03B 5/58; B03B 11/00; G05D 23/19; G06F 1/20; G06F 1/3203; G06F 1/3206; G06F 1/3209; G06F 1/3228; G06F 9/45558; G06F 9/4893; G06F 9/5094; G06F 13/409; G06K 19/0723; G06K 19/07705; G06K 19/07722; H04L 69/329; H05K 7/20836; G06F 2009/4557; Y02D 10/00

(57) ABSTRACT

A system, and method for controlling a computing system, comprising: reading a stored energy-performance characteristic of a plurality of different phases of execution of software, an execution of each phase being associated with a consumption of a variable amount of energy in dependence on at least a processing system performance state, the performance state being defined by a selectable performance-energy consumption optimization for at least two processing system components; scheduling a plurality of phases of execution of the software, in dependence on the stored energy-performance characteristics, for each of the respective phases of execution of the software and at least one system-level energy criterion; and executing the phases of execution of the software in accordance with the scheduling.

### CROSS REFERENCE TO RELATED APPLICATIONS

The present application is a
Continuation of U.S. patent application Ser. No. 18/425,536, filed Jan. 29, 2024, now U.S. Pat. No. 12,510,943, issued Dec. 29, 2025, which is a
Division of U.S. patent application Ser. No. 17/473,107, filed Sep. 21, 2021, now U.S. Pat. No. 11,886,914, issued Jan. 30, 2024, which is a
Continuation of U.S. patent application Ser. No. 15/490,525, filed Apr. 18, 2017, now Abandoned, which is a
Continuation of U.S. patent application Ser. No. 12/841,160, filed Jul. 21, 2010, now Abandoned, which
Claims benefit of priority from U.S. Provisional Patent Application No. 61/227,361 , filed Jul. 21, 2009, each of which is hereby expressly incorporated by reference in their entirety.

### FIELD OF THE INVENTION

The present invention relates to the field of server energy and cooling management.

### BACKGROUND OF THE INVENTION

The data center energy crisis has been in the making for the past several decades, as data centers are designed primarily with peak performance and peak capacity in mind. With the doubling of transistor counts and performance in semiconductor devices at 18-month intervals following Moore's law, energy dissipation in servers have grown at an alarming rate. The smaller form factors of modern blade servers have, at the same time, permitted more and more servers to be packed into a given physical space, further worsening the already critical situation with server power dissipations within data centers. Adding to all of this is the trend to overprovision data center capacities and the use of overrated power supplies for the individual servers. Such over provisioning results in gross energy inefficiencies as servers and power supplies are generally designed to give very high energy efficiencies only at or near peak loading levels. The net result of all of these is that 50% and upwards of the total cost of ownership (TCO) for a data center is in the utility costs of operating and cooling the servers. From an economic standpoint, we spend about 2% of the nation's annual energy consumption on data centers. With electricity costs growing annually at about 7% to 10%, the situation is bleak and needs immediate correction with the use of innovative and dramatic solutions. The other benefits of operating energy-efficient data centers are of no less significance - reducing the carbon footprint and making the nation energy-secure are also worthy goals.

Traditional approaches to managing the data center energy crisis have been to use advanced cooling and packaging solutions, to use DC power sources for servers and a variety of other solutions at reducing the energy dissipation within servers. These latter solutions have included the use of dynamically changing the power-performance settings for individual server components, such as processors and hard disk drives, or on policy-based job scheduling that schedule the offered workload across servers to meet thermal objectives. The growing use of virtualization technologies in data center also supports flexible scheduling-based energy management schemes. Virtually, all of these solutions are reactive in nature: energy management or cooling solutions are adjusted based on feedback from sensors that sense temperature or some activity parameter (such as current computing load, performance metrics).

### SUMMARY OF THE INVENTION

The present technology assumes, according to one embodiment, a holistic view of data centers as a cyberphysical system where the cooling solutions work in unison with the computing level solutions for energy management in a coordinated fashion. The total energy expended in the computing components and the energy expended in the cooling system is treated as a first-class resource that needs to be scheduled explicitly to maximize the overall energy-efficiency of the data center. One embodiment of aspects of the technology is multi-tiered and includes:

The use of fast models for predicting local and global thermal conditions to promote overall energy efficiency. The thermal models, in turn, are driven by empirical models of energy dissipation within servers and switches as a function of the measured values of a variety of actual activity counts. This approach of jointly using accurate energy dissipation models for the computing equipment and fast thermal models permit the cooling solutions (adjustment of inlet temperature, air flow speed and pattern) to be proactive.

The use of a global scheduler to allocate individual energy budgets to servers as a function of the workload, the predicted thermal trend, actual server utilizations and temperature and airflow measurements from sensors. The cooling efforts are also matched to the predicted thermal trends and are rack specific, instead of being rack agnostic, as in traditional systems. Alternatively stated, the cooling efforts for a rack are directed, dynamic and matched to the thermal conditions in the rack's environment. This results in the most energy-efficient use of the cooling resources.

The use of modified server operating system kernels that permit the individual servers to stay within their assigned energy consumption budget. Software solutions at the operating system kernel level exercise existing power management actuators inside the processor and other components of servers in a proactive fashion to stay within the dictated energy budget and in a reactive fashion based on the thermal condition of its environment. Thus, the system uses a predictive model of the thermal conditions based on analysis of a set of “tasks” or other prospective activities, as well as a feedback driven control which employs sensors or indicia or actual conditions. The predictive model may be adaptive, that is, the predictive model may be modified in dependence on the actual outcomes as determined by the sensors or indicia. In addition to the sensor or indicia inputs, the system may also receive a price or cost input, which permits a price or cost optimization, rather than an efficiency optimization. By imposing an external price or cost consideration, the system can be made responsive to peak energy demand considerations, and also a prioritization of tasks, which may each be associated with a task value.

Each of these technologies may be employed together, separately, or in subcombination. The thermal models, for example, can be implemented with minor modification to semiconductor devices, to provide software access to registers and counters which monitor operation of the chip. As the chip processes information, various types of activities are tracked, and these tracked activities may then be read by software to implement the models. The models may be executed on the same semiconductor as an additional process within a multitasking processing stream, within a special core dedicated to this process, either on or off the integrated circuit, or by a remote system. The modified server operating system kernels typically do not require hardware modifications, though sensors may be required beyond those present in standard components of the computing system. In particular, integration and interfacing of external cooling system sensors and controls may require additional hardware modules. The global scheduler is typically provided as part of a load distribution switch, which is a standard hardware component, but executes software in accordance with the present embodiments. In particular, the task allocation algorithm favors loading of servers to near capacity, which may be defined by performance limitations or thermal limitations, before allocating tasks to other servers. The allocation may distinguish between different blades within a rack, with each rack typically being controlled on a thermal basis, i.e., to stay within a desired thermal envelope while achieving cost-efficient cooling, while each blade may be allocated tasks which balance performance and energy efficiency, while remaining within safe thermal limits.

The net result of a combination of all of this is a control system that uses a combination of proactive and reactive elements in a multi-tiered strategy for co-managing the thermal and computing solutions for promoting the energy efficiency (or cost effectiveness) of the data center. However, these technologies need not be employed together to gain benefits. Likewise, the chip, operating system (software), and system level optimizers need not communicate with each other, though they are preferably aware of the multilevel optimizations, which may alter responses to conditions. For example, a prediction of and control over future processing load must be coordinated between the various system levels in order to avoid conflicting efforts or over-compensation.

A preferred embodiment may be implemented in a scaled down data center consisting of Linux server racks with floor plenum and portable computer room air conditioners (CRACs) and a variety of sensors, or a full data center with server racks in a facility with centrally or distributed control cooling system. Preliminary results indicate that the present approach can realize about a 20% improvement in the energy efficiency of the data center.

With the growing concern about energy expenditures in server installations, mechanisms are sought for improving the energy-efficiency of server installations. Jobs running on a typical data or compute server are quite diverse in their energy-performance requirements. The present technology provides a system and method for dynamically configuring a server system and its associated job scheduling strategy to elevate the energy-efficiency of the server system.

Typical servers have a set of “gears”, i.e., performance settings which optimize performance in one or more ways, with “cost”, such as energy cost, thermal rise, or other metric. These settings are available to permit an administrator to choose the most energy-efficient setting for realizing a specific performance goal to match a specific type of application running on the server. The settings can control the processor, the dynamic random-access memory (RAM), the network interface and associated software and the secondary storage system-individually or in combination. For example, a system administrator may program clock rate of a computer, select RAM performance based on wait states, and implement other performance tweaks. In general, higher performance settings have a higher energy cost, both in the unloaded state and the fully loaded state.

In systems having alterable performance settings, one limiting factor is often maximum system component temperature, which in turn is impacted by heat dissipation. Therefore, by establishing performance settings which are near those minimally necessary to achieve suitable results, the system may be able to achieve higher throughput, since the power budget is better distributed to those aspects which are in most need, and not wasted on excess component performance which does not add to task-normalized system performance.

A technique is provided for learning the energy-performance characteristics of jobs or phases of jobs as they execute within a computing system. This information is used to optimize the scheduling of new jobs as well as repetitive jobs with the server characteristics set dynamically to meet the performance demands using the most energy-efficient configuration for realizing the performance goal. The technique also optimizes the energy and performance overhead for dynamically changing the energy-performance setting of the server.

A mechanism for classifying entire jobs or phases of individual jobs into different classes, where each class is associated with a unique performance gear setting is provided. The job or job phases are classified into at least the following categories: compute bound, memory bound, network bound, and secondary storage bound. This classification technique relies on statistics maintained in hardware and the operating system on a typical server. This classification is performed dynamically as the job executes. A single performance setting or a set of performance settings are associated with each such class. Job identities and their classifications may be maintained in a tabular form within the server system. Alternately, a process control block or other data structure may be employed.

Jobs submitted to servers are often very repetitive in nature. When such jobs are encountered, their class can be looked up from the aforesaid table. In one embodiment, queued jobs belonging to the same class are scheduled back-to-back using a performance setting appropriate for the class. Such a scheduling strategy minimizes the energy overhead and performance penalty that would normally arise from changing the gear settings from a job to the next. The same scheduling strategy can be applied to individual phases of a job: the scheduler schedules job phases of different jobs back-to-back, where the phases belong to the same class. A further refinement of this technique will be to have each class divided into subclasses, with a unique performance setting for each subclass. Unclassified jobs or job phases are scheduled by making some default assumptions about the job class; this can change as the job executes-based on its observed behavior.

In an alternate embodiment, complementary tasks are allocated to a single set of resources for execution, such that the available capacity of the server under the specified performance settings is efficiently utilized. For example, one task may incur significant loads on processor and memory, while another on hard disk. By combining these tasks for concurrent execution, each proceeds with high efficiency.

However, it is noted that by tuning the performance of the system to a single task or type of task, in some cases a higher energy efficiency is achieved than in heterogeneous processing environments, even where the task allocations are balanced.

One embodiment of this technology provides that the task allocation is optimized by a software application running on the hardware whose energy efficiency is to be optimized while maintaining acceptable performance, for example an operating system, and that the scope of the optimization is the hardware under control of the software infrastructure.

This technique thus has the potential for improving the energy-efficiency of server systems through relatively modest changes in the operating system kernel. No reliance particular is made on unique hardware or dedicated hardware support.

In order to calibrate the system, a test suite of programs may be provided which exercise different performance aspects of the system. One or more sensors may be provided to monitor the results, which is for example a temperature. In some cases, system components have integral temperature sensors, such as processors, hard drives, memory modules, mother boards, fans, cases/enclosures, and other hardware components, and the operating system may read these sensors. In other cases, sensors are added to the system. Thus, the system may be calibrated for thermal output/performance under different task loading conditions. For example, a multidimensional table or other data structure. may be created from this calibration data, and this may be used as a lookup table during operation, or the table processed to generate a compact algorithm which predicts thermal performance from task characteristics. When the system is running arbitrary tasks, the operating system may initially seek to classify the task based on certain characteristics. The characteristics are then used to query the lookup table, or exercise the compact algorithm, which results in an initial allocation of the task and specification of performance settings, or in the case of a system encompassing multiple variants with different performance criteria, an allocation of the task to the selected system. While the live task is running, the predicted performance may be compared with the actual performance, and the system corrected. In general, the correction will be applied to the code module or a signature or key features of the code module being executed, since the table itself is established based on calibration data. Of course, a system may also avoid a calibration phase, and construct a table or algorithm exclusively from live data.

In order to optimize the system performance, a genetic algorithm or other self-tuning algorithm may be implemented, in which during operation, or between successive operations, parameters are changed to “search” for a more efficient operating point, or otherwise to test the effect of various changes in performance and efficiency. Over time, the control system, which is, e.g., the operating system of the computing system which controls task initiation and allocation, will learn, at any existing operating point of the system, how an incremental task of a characterized type is most efficiently performed. A variety of options may be available, including waiting until a future contingency is satisfied, distributing the task to the resources that are predicted to permit completion soonest (i.e., the task runs with highest urgency), distributing the task to existing resource sets with particular performance profiles, and modifying the performance profile of a set of resources on which to execute the task.

As discussed above, a penalty may be incurred in altering the performance profile of a set of resources. Further, a set of resources may be used to process a plurality of tasks, having a range of optimal hardware performance characteristics. Therefore, adjusting the performance profile ad hoc to simply optimize for the new task is disfavored, unless the performance profile is suboptimal for a predicted significant set of tasks, such that the alteration in performance characteristics produces a net benefit.

If the system as a whole is thermally limited, then there is a net benefit to executing tasks in an power dissipation efficient manner, with the entire energy consumption of a task considered as a unit. Often, one or more components of the system will also be thermally limited, e.g., the processor core(s). Therefore, in addition to allocating tasks in an energy efficient manner, tasks must be allocated which do not exceed a component thermal limit, and this may entail allocating some portion of tasks to the system which are relatively inefficient on a system basis, but produce less stress on a limiting component.

It is noted that the tasks need not be characterized by the operating system at run time. Rather, the compiler or another system may produce an in-stream communication, or parameter file associated with the executable code, that defines a type and target performance environment of execution. The operating system can then seek to allocate the task to hardware with the target performance profile configuration. In general, the reference optimum platform may be different than the actual optimum. For example, the actual platform may have extraneous tasks executing, have a different thermal dissipation profile or different component selection, or otherwise deviate from the reference design. Therefore, empirical data is preferably employed, even if the program code is pre-characterized.

The parameter space for the system, outside of the code itself, includes for example, the ambient temperature (facility, external), energy cost (which may vary diurnally), peak demand issues, cooling system efficiency (air conditioning or water cooling), air flow rate, air flow dynamics, average system temperature, peak component temperatures, thermal impedance, fan speed(s), etc. In fact, there may be sufficient factors that explicitly compensating for them may be difficult or impossible, and indeed the system may include intrinsic responses that may defeat external controls or presumptions of a static system. For example, many components of modern computing systems have internal thermal sensors, and firmware that is thermally responsive. Therefore, the response to changes in temperatures, especially near peak loads or peak temperatures, may be non-linear and somewhat unpredictable.

The operating system preferably seeks to profile the code or module to be executed, and allocate the code or module to a processing system having one of a predefined class of performance settings, e.g., four different classes, e.g., processing centric, memory centric, disk centric and network centric. Additional tasks are typically allocated in accordance with their classification, but if and when the tasks on a system deviate from the nominal, the task scheduler may allocate compensating tasks seeking to renormalize the mix of tasks. In this way, a multiprocessing system can gain efficiency, since each system operates near its peak efficiency, which is higher than the efficiency of a general purpose system executing random code segments. If the mix of code changes, in some cases the system may be modified to assume a different performance profile.

It is therefore an object to provide a method for controlling a computing system, comprising: reading a stored energy-performance characteristic of a plurality of different phases of execution of software, an execution of each phase being associated with a consumption of a variable amount of energy in dependence on at least a processing system performance state, the performance state being defined by a selectable performance-energy consumption optimization for at least two processing system components; scheduling a plurality of phases of execution of the software, in dependence on the stored energy-performance characteristics, for each of the respective phases of execution of the software and at least one system-level energy criterion; and executing the phases of execution of the software in accordance with the scheduling.

It is also an object to provide a computing system, comprising: at least two processing components, each having a selectable performance-energy consumption optimization; a memory configured to store energy-performance characteristics of a plurality of different phases of execution of software, an execution of each phase being associated with a consumption of a variable amount of energy in the computing system in dependence on at least a processing system performance state, the performance state being defined by the selectable performance-energy consumption optimization for at least two processing system components; and a scheduler, configured to schedule a plurality of phases of execution of the software on the at least two processing components, in dependence on the stored energy-performance characteristics, for each of the respective phases of execution of the software and at least one system-level energy criterion.

The energy performance-characteristic may be supplied with the software to the processing system. The energy performance-characteristic may also be determined empirically by monitoring an execution of the software phases.

A plurality of software phases executed concurrently, or sequentially.

A plurality of software phases of the same or different software tasks, having similar or complementary energy-performance characteristics, may be scheduled in sequence.

A stored energy-performance characteristic of a software task, comprising a plurality of software phases, may also be read and utilized. The scheduling may attempt to schedule a plurality of software tasks having a similar energy-performance characteristic back-to-back.

It is a further object to provide a computing system, comprising: a memory configured to store energy-performance characteristics of a plurality of different phases of execution of software; a plurality of processing components, each having a selectable performance-energy consumption tradeoff, an execution of each software phase on the plurality of processing components being associated with a consumption of a variable amount of energy in dependence on at least the selectable performance-energy consumption tradeoff; and a scheduler, configured to schedule a plurality of software phases on the plurality of processing components, in dependence on the stored energy-performance characteristics and at least one system-level energy consumption-related criterion.

The scheduler may schedule tasks in dependence on at least a similarity of classification of an energy-performance characteristic of a phase with respect to a prior scheduled phase.

### BRIEF DESCRIPTION OF THE DRAWINGS

FIG. 1 depicts the control system aspects of the present data center management strategy.

FIG. 2A depicts the state of affairs in prior art servers and shows how the power dissipation and energy efficiency of a typical server varies with server utilization.

FIG. 2B depicts the intended overall impact of the present solution on server power dissipation and server energy efficiency plotted against server utilization.

FIG. 3 shows a block diagram of a prior art computing system.

### DETAILED DESCRIPTION OF THE PREFERRED EMBODIMENTS

According to a prototype embodiment, a scaled down data center is provided which demonstrates a unique approach to addressing the data center energy crisis. The energy spent on the computing equipment and by the cooling system is treated as a first-class resource and managed explicitly in the present approach in a proactive as well as reactive manner. Instead of the traditional approach of cooling the server racks uniformly, dynamic and directed cooling is employed, that skews the cooling efforts to match the actual and projected cooling demands of the individual or groups of racks. Cooling for a rack is controlled based on sensors (i.e., a reactive control), a prospective set of tasks or functions in a queue (i.e., a proactive control), and an operating system component of each subsystem which permits a modification of energy demand.

It is noted that a cooling system may have higher efficiency when cooling a relatively hotter server than a cooler one, and therefore overall efficiency may be increased by permitting some server racks to run near a maximum operating temperature, and other racks to be essentially deactivated, pending peak demand recruitment. While running at relatively higher temperatures may be a factor in reducing a mean time between failures (MBTF), the usable life of blades in a data center is typically well in excess of the economic life; further, even if there is a failure, the data center will typically have automatic failover fault tolerance systems. Indeed, if some racks in the data center are specifically designed to always run near peak capacity and high temperature, these may be configured for more efficient operation, for example, greater spacing from other racks, to permit better heat load shedding without affecting adjacent racks, and higher temperature specification components.

It is also noted that in some cases, it is not the temperature per se which adversely impacts the MBTF of a system, but rather the thermal cycling and mechanical stresses on components, circuit boards, and packaging. In such cases, the operation of a rack at a consistent hot temperature may be an advantage over a system which seeks, for example, a uniform minimum temperature of all racks which varies with data center load.

One embodiment of the technology improves the overall energy-efficiency of a data center in a holistic manner, and targets both the energy expended in operating the equipment and the energy expended in the cooling system. A key aspect of is to coordinate the activities of all of the energy consumers in a data center. These consumers include the individual severs and communication infrastructures as well as the cooling system components. Some current solutions to this problem have addressed inefficiencies in the use of power conversion devices, the cooling system and the servers themselves [Sh 09, BH 07, BH 09, LRC+08]. Emerging solutions to this problem have also started to address the need to coordinate the activities of these consumers [BH 09, NSSJ 09, SBP+05, TGV 08]. As an example, the work of [TGV 08] has proposed an approach for minimizing the energy expended on the cooling equipment by minimizing the inlet temperature through appropriate job scheduling. The work of [NSSJ 09] coordinates the energy expended on the computing equipment and the cooling infrastructures and allocates energy budgets to virtual machines. Such VM energy budgets are not easy to implement, as energy expended by a VM is not easy to track and control; energy dissipation in many related components are ignored in simplifications that are used. In general, emerging solutions have a number of potential limitations:

The energy and performance overhead associated with job rescheduling and VM management and server-local scheduling overhead are ignored. The communication infrastructures within a data center are heavily utilized and are prone to congestion, resulting in significant added energy dissipation if jobs are rescheduled.

A simple rescheduling of the jobs may not make the most energy-efficient use of the servers and racks—the operating configurations of such servers have to be continuously adapted to fit the characteristics of the workload.

Simple reactive control systems, as proposed in all existing and emerging solutions, do not address the problem of thermal lags and delays associated with temperature sensors, whose inputs are used by the actuators in these systems.

The implicit assumption in most current systems that that all servers and racks have a uniform external cooling requirement may not be the best one for improving overall energy efficiency. While we do have some proportional cooling facilities in the form of automatically adjusted CPU cooling fan and enclosure fan speeds, external cooling systems are generally uniform and oblivious of the specific cooling needs of an entire rack. In general, higher energy efficiency will result by redirecting additional cooling to regions that can benefit from it, resulting in a dynamic, directed cooling system.

The present approach allocates energy budgets to servers, racks, storage and communication components and adapts the cooling effort dynamically to match the energy dissipated in these components. The energy consumption in the computing components are modeled using accurate empirical formulas and server-local (and global) scheduling techniques are used to limit server energy consumption within the allocated budget. This is a far more practical approach compared to any scheme that operates on the basis of energy budget allocations to VMs. The energy dissipation estimates from these empirical models are used to schedule the energy budgets for the computing equipment and the dynamic cooling system, along with the workload. Last but not the least, the present control system uses both proactive and reactive control mechanisms to manage the data center effectively in the face of sudden workload variations and to mitigate latencies associated with the activation and deactivation of servers and VMs.

In current data centers, the software systems infrastructures (including the Linux OS and popular file systems) are very limited in their adaptation capabilities in this respect. The most popular mechanism used for adaption is dynamic voltage and frequency scaling (DVFS) on the processing cores, and other components of the computing platform are unaddressed. This is not a desirable situation from the standpoint of energy efficiency, as the total of the energy dissipations within the DRAM modules and in the backplane and other communication infrastructures is about 45% of the total energy expended by a server, while the processors consume about 30% of the total energy [BH 09]. Current measurements seem to indicate that the processor energy dissipation will continue to decrease relative to the energy dissipation within the other components of a server [BH 09]. At the server level, it is thus critical to incorporate mechanisms that address the energy dissipation across all major components of a server instead of just focusing on the processing cores.

At the data center level, the energy expended in the communication infrastructures (switches, in particular) and in the cooling system itself should be considered. The present approach considers the total energy expended in the computing, storage, communications and cooling system as an explicitly scheduled resource and to schedule the computing and cooling resources using a common framework. The end goal is to maximize the energy efficiency of the data center, consistent with the performance goals. As discussed above, a cost optimization paradigm may also be implemented. In a cost optimization, the costs and benefits are normalized, and a set of conditions with a maximum net benefit is selected. The costs in this case may be energy costs, though other costs can also be considered in the calculation, such as maintenance costs, operating costs, license fees, etc. The benefits are typically considered as the net work output of the system, e.g., computing results, though values may be placed on the speed, latency, accuracy and completeness, etc. of the result. Indeed, assuming the same computational task, the result may be worth more to some users than others. Thus, the energy efficiency considerations may be modified or distorted based on a variety of extrinsic factors. The cost optimization factors may be analyzed in a centralized controller, which permits an allocation of tasks at a scheduler or load balancer element, distributed to the various processing cores and made part of the modified operating system kernel, or a hybrid approach. Of course, other elements may also provide these functions.

### Example Use: Integrated, Dynamic Management of Computing and Cooling Resources

The system preferably makes the best use of the energy expended in operating the computing and communication equipment as well as the energy expended in the cooling system. The energy expended by the computing and communication equipment and the cooling system is considered a first-class resource and managed explicitly. Servers are allocated individual energy budgets and a modified Linux kernel in the servers is used to dynamically adjust the system settings and perform a local scheduling to stay within the individual server's energy budget allocation. The computation of the energy budgets for servers/racks and the control of the cooling system to effectively define a thermal envelope (that is, cap) for each server/rack for is done by a global module that senses a variety of conditions, as described later, to direct global job scheduling and to control the cooling system components, skewing the cooling effort across racks and regions as needed to improve the overall efficiency of the cooling system.

Another distinguishing feature of a preferred embodiment of the system is in its use of three controls for adapting a cooling system: the air flow rate directed at the racks from portable CRACs, the inlet temperature and the use of movable baffles to redirect air flow. Traditional solutions have largely looked at one or two of these adaptation techniques (mostly inlet temperature and somewhat rarely, air flow rate).

Using the terminology of [RRT+08], the integrated data center management technique is essentially a control system with the following components critical to the management:

Sensors: On the thermal/mechanical side, the sensors monitor the temperature and air flow rates in various parts of the rack and the room. On the computing side, the sensors are in the form of hardware instrumentation counters within the processing cores, counters for device and system utilizations maintained by the operating systems, variables that record the incoming queue size and others.

Actuators: The management policy exercises various actuators to adapt the cooling system and the servers. On the thermal/mechanical side, the actuators adjust fan rates for regulating the air flow from CRACs, operate servo motors to adjust the baffles for air flow direction and use electromechanical subsystems to adjust the inlet temperature. On the computing side, the software elements used as actuators (a) control the voltage and frequency settings of the cores and activate/deactivate individual cores to ensure that they do not exceed their allocated energy budget and to respond to thermal emergencies at the board/component level; (b) schedule ready processes assigned to a server and adjust core settings (using (a)) to maximize the energy efficiency of the server; (c) perform global task scheduling and virtual machine activation, migration and deactivation based on the dynamically computed thermal envelopes and rack/server level energy budgets.

Controllers: The control policy itself will be comprised of two parts; the proactive and reactive, which are described in detail below.

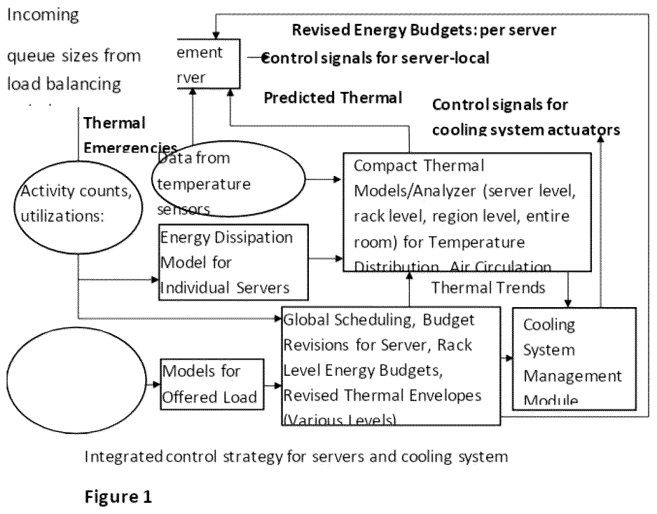

FIG. 1 depicts the control system aspects of one embodiment of a data center management strategy. This control system uses a combination of proactive and reactive strategies:

Proactive strategies: two different types of dynamic proactive management of data centers are provided. These are:

Because of thermal lags, temperature sensors are unable to detect the onset of thermal emergencies due to sudden bursty activities with the server components, including those within the DRAM, cores, local (swap) disks, if any, and the network interfaces. Empirical power models for the server energy dissipation are therefore derived, using activity counters maintained within the Operating System and the built-in hardware instrumentation counters, as described below. The estimate of the energy dissipation of an individual server is based on sampled estimations of the activities (similar to that described in [PKG 01]). This estimate of the energy dissipated by a server within a sampling interval is used to guide local scheduling and control the local system settings. The estimates of the server energy dissipations within a rack are also used as the inputs to a fast, optimized and calibrated thermal model that provides data on the thermal trends, taking into account the environmental conditions. The computed thermal trends are used, in turn, to guide global and rack level job scheduling and VM management as well as to proactively direct cooling efforts towards a region of rising temperature/hot spot.

The front end queues of the switches used for load balancing are a good indicator of the offered computing load to a server. These queues are therefore monitored to proactively schedule new jobs in a manner that improves the overall energy efficiency of the data center. This proactive monitoring of the input queue also permits absorption of some of the latencies involved in activating racks and servers that are in a standby mode, as well as to absorb some of the latencies in VM migration. In fact, as described below, the proactive monitoring of the incoming queues of the load balancing switches also permits activation/deactivation and migration of VMs, taking into account the energy overhead of such management.

Reactive Strategies: The reactive strategies include the following sub strategies:

A management system to ensure that the energy consumption of the individual servers does not exceed their dictated energy budget. This subsystem controls the computing components as well as the network interface. This management system is part of the modified Linux kernel of the servers that uses a server power estimation model and the sampled value of the instrumentation counters found in modern microprocessors and other statistics maintained by the kernel to control system settings (including the DVFS settings).

A subsystem within the kernel that reacts to local and neighborhood thermal emergencies or trends, as detected from local/neighborhood temperature sensors as well as information generated by the fast thermal models/analyzer to either shut down individual servers/racks or to reconfigure server settings to reduce their energy dissipation. This subsystem is an added protection mechanism that works in conjunction with the other energy management systems—both reactive and proactive, and deals with high-impact unexpected emergencies such as CRAC failures.

In conjunction with (2) above, a subsystem that monitors the local/neighborhood thermal trends to allocate and direct local cooling capacity in a focused fashion to minimize energy consumption from the cooling system. This will operate on a slower time scale than the computing reactive strategies. The computing approach of (2) above and this thermal systems approach should operate synergistically to minimize the overall global energy usage while maintaining compute performance. The reactive controller will constantly tradeoff energy minimization between the computing and thermal systems activities.

A subsystem within the global budgeting module that reacts to global thermal emergencies based on sensed environmental conditions in the room and trend data computed by the fast thermal model/analyzer.

A subsystem within the global budgeting module that reacts to the data on actual server/rack utilizations to throttle down servers/racks as needed.

The overall goal of all of the control system elements, both proactive and reactive, is to maximize the overall system performance under the energy constraints dictated by the budgeting module. The budgeting module ensures that the relative components of the energy dissipated by the computing/communication elements and the cooling system are optimal.

### Server Management

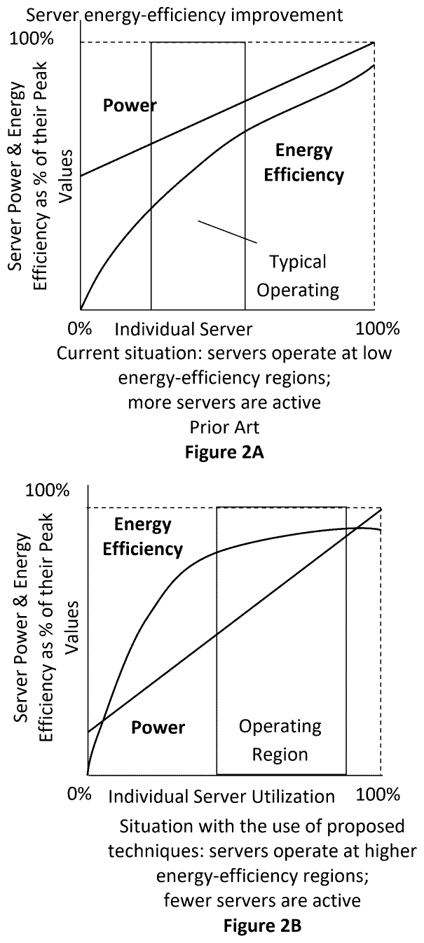

The goal of our proposed effort is to improve the overall energy efficiency of the servers and the cooling system. To do this, we attempt to minimize the number of active servers and operate them at or near their peak loading level to maximize their energy efficiency. The existence of virtual machine support certainly makes this approach practical. At the same time, we minimize the energy consumption in the cooling system by just providing sufficient cooling for the active servers. FIG. 2A depicts the state of affairs in current servers and shows how the power dissipation and energy efficiency of a typical server varies with server utilization. As seen in FIG. 2A, the energy-efficiency is quite low at low server loading (utilization) and the power dissipation remains relatively high. FIG. 2A also depicts the typical operating points of servers—the typical average server loading is significantly lower than the peak loading—as a result, the overall energy efficiency is quite low at these typical operating points.

FIG. 2B depicts the intended overall impact of the present technology on server power dissipation and server energy efficiency plotted against server utilization. The present multi-tiered server power management technique (which subsumes standard power management techniques) improves the server energy efficiency dramatically and simultaneously reduces the power dissipation at lower server utilization levels. The overall server efficiency thus remains quite high at the typical load levels and across a wider range of loading, as shown in FIG. 2B. Second, by globally scheduling more work to a fewer number of active servers (and by keeping the non-active servers in a standby state), we push the workload level on individual servers more towards a region where energy-efficiency is very high. The expected result of all of this is a solution that, based on a quick back-of-the-envelope calculation, can enhance the overall energy efficiency of servers by about 15% to 25% on the average beyond what is provided by the state-of-the-art, even when the added overhead of the present solution is factored in. Improvements in power savings are expected to be similar. One down-side of operating servers at or near their peak capacity is that any sudden changes in the behavior of their assigned workload can cause switching activities to go up and lead to local thermal emergencies.

In general, servers can be more efficiently managed than presently feasible if they:
R1) Have mechanisms to put a hard limit on server energy dissipation to avoid thermal emergencies.
R2) Have a proactive mechanism to activate or deactivate virtual machines or servers or entire racks to match the offered load taking into account any energy and performance overhead for activation and deactivation.
R3) Have techniques that implement a more energy-proportional relationship between server power dissipation and the server utilization, as shown in FIG. 2B.
R4) Extend the operating region over which a server has high energy efficiency: this permits higher server energy efficiencies even at moderate load levels.

The implementation of requirements R3 and R4 lead to the situation shown in FIG. 2B. The approach to implementing these requirements in software, on existing systems, is now described.

### Implementing the Requirements R1 Through R4

Empirical energy dissipation models are preferably used to determine the energy consumed by a server and this estimate is used to cap the energy consumed by a server. This approach is adopted since it is not practical to use external power meters on each server to determine their energy consumption.

Empirical models for the energy dissipated by a server have been proposed in the past; the simplest of these models are based on the use of utilization data maintained by the operating system (such as core utilization, disk utilization) and is, for example, of the form:

$${P}_{server} = {K}_{0} + {K}_{1} \times {U}_{proc} + {K}_{2} \times {U}_{mem} + {K}_{3} \times {U}_{disk} + {K}_{4} \times {U}_{net}$$

Of course, other, more complex forms, may be employed.

Where the Ks are constants determined empirically and the Us refer to the utilizations of the processor (U_{proc}), memory (Uₘₑₘ), the disk(s) (U_{disk}) and the network (Uₙₑₜ). The operating system maintains and updates these utilization data. As reported in [ERK+08], the actual measured power and the power estimated from the above equation are quite close and typically within 10%. A recent effort extends simplistic models of this nature to regression based predictive models that predict server energy consumption on long-running jobs as a function of the core energy dissipation, L2 cache misses and ambient temperature [LGT 08]. The model of [LGT 08] is a good starting point for our efforts. We will extend this model with additional metrics obtained from hardware instrumentation counters found in typical cores as well as slightly modified system calls for network/file I/O to account for energy dissipation within network components to accurately account for remote data access and inter-process communications and I/O activity (which were ignored in the work of [LGT 08]).

To track and predict the energy consumption of servers in software, sampled measurements of the hardware instrumentation counter values and OS-maintained counters for computing utilization will be used, in manner reminiscent of our earlier work of [PKG 01]. The modified thread scheduler in contemporary Linux kernels will use these sampled measurements to guide local scheduling within a server so as to limit the server energy consumption within a sampling period to stay within the limit prescribed by the global energy/workload scheduler. In addition to the traditional DVFS adjustments, the behavior of threads within the sampling periods will be classified as CPU bound, disk bound and network bound and schedule similar threads back-to-back to avoid unnecessary changes in the DVFS settings (and avoiding the energy overhead and relatively long latencies in changing such settings). This in turn addresses Requirements R3 and R4. The modified scheduler will also react to thermal emergencies as detected by external temperature sensors (which are read and recorded periodically by the scheduler itself on scheduling events within the kernel).

Requirement R2 is implemented in the global scheduler, as described below, by keeping track of the workload trends (through monitoring of the incoming request queues at the load balancing switches) and job completion statistics. If the global scheduler sees a growth in the job arrival rate, it activates VMs/servers/racks as needed to cope with the additional workload. The overhead for such activation and deactivation, including the energy costs of moving VM contexts are accounted for in this process, and thus requirement R3 is also addressed.

Techniques for message consolidation that packs several short messages into a single message within a jumbo Ethernet frame within the network interface to amortize the flat component of per-packet overhead of network transfers may also be employed. This also addresses Requirement R3.

A different way of amortizing the scheduling overhead (including the changing of the DVFS settings of cores) exploits the characteristics of repetitive jobs. In a typical server installation, the number of such jobs is expected to be quite high. For example, repetitive jobs of the SPECweb 2006 benchmarks on a Linux platform (with Intel E5460 cores) running Apache were dynamically classified into two classes: compute bound and I/O bound, based on utilization statistics maintained by the kernel and instruction commit rate data maintained in the hardware instrumentation counters. This classification data was maintained within the Apache server. Jobs of the same class in the work queue of Apache were scheduled back-to-back wherever possible and the DVFS settings of the dual core platform were explicitly controlled. Unnecessary changes in the DVFS settings were also avoided and job wait times on the queues were limited to maintain a performance level close to that of the base case. The CPU power measurements (made with a power clamp on the power cord for the core going from the power supply to the motherboard) showed that this simply strategy reduced the core power consumption by about 11%.

For the present system, this technique can be moved to the kernel level for added efficiency, extend the classification to add memory bound jobs (jobs that trigger a high proportion of RAM activity, as evidenced by the on-chip cache miss instrumentation counter) and network bound job classes, for instance. This classification information is used to schedule jobs that match the characteristics of processor sockets with a preset independent performance or to cores within a multicore chip that permits the use of similar preset performance settings independently for each core. The preset performance settings are changed only under load increases that saturate the capacity of a core with a specific DVFS setting. This approach of exploiting pre-classed job addresses requirements R3 and R4 simultaneously.

### Global Energy Budget Allocation and Workload Scheduling

The global scheduler (GS) of a preferred embodiment of the system is responsible for the allocation of energy budgets for the VMs/servers/racks and the assignment of workload to the individual machines. The key requirement of the GS is that it has to be fast and scalable. The GS may be implemented on a few dedicated multicore machines which also implement the compact thermal analyzer and models. Multiple machines may be used to permit scalability; for a small server installation, it may be possible to implement all of the functions on a single multicore platform. These dedicated machines may also receive data from a variety of sources, which are optional, as shown in FIG. 1.

The GS maintains a variety of tables that record the energy/performance characteristics of each rack, its utilization statistics, and data on the environmental temperature computed from various sources. The GS also maintains a list of quality of service (QoS) requirements (guaranteed transaction rates, data delivery rates etc.) for implementing differentiated services. The GS also senses the incoming work queue sizes at the load balancing switches and uses simple workload models to predict the impact of incoming workload. The simple workload models can simply classify incoming jobs based on the request types or use more sophisticated information about pre-classified repetitive jobs. The GS schedules the workload to maximize the workload allocated to active servers/racks, assuming VM support on all nodes. This allocation uses the thermal data—obtained from the compact model as well as from thermal sensors and using service guarantees as a constraint. Cooling requirements and changes to the energy budget for the computing/storage and communication equipment for the allocated workload are also assigned based on a variety of heuristics. Some possible heuristics include (but are not limited to):

Extrapolate the thermal output of the active servers and revise its energy budget and cooling requirement based on the updates to number of jobs (existing plus newly-assigned) assigned to the server.

Use the energy requirement characteristics of known, repetitive jobs and the heuristic above for unclassified jobs to plan the schedule.

Use the data maintained on the average job completion rate and average energy requirement of jobs to guide the allocations.

As mentioned earlier, the GS keeps track of the job dispatch rate and the size of the incoming queues in the front-end load balancing switches to keep track of the workload trend. This trend data is used to activate or deactivate servers and racks and redirect cooling efforts as needed. The energy expended in such activation/deactivation and in migrating VMs, where necessary is accounted for in the allocations.

Alternative scheduling may also be employed, including ones that dynamically switch scheduling strategies based on the thermal conditions and current workload. As an example, if all servers are being operated in the high energy-efficiency region as shown in FIG. 2B, then it may be better to perform an allocation that balances the load across the racks to avoid the formation of hot spots in the server room.

The GS has similarities with data center configuration systems and managers from several vendors (e.g., IBM's Tivoli suite) [IBM 08a, IBM 08b]. However, the present system differs from these schedulers in at least the way server energy dissipation estimates are made at a finer granularity, in making use of a thermal model to predict and cope with thermal conditions, and in using dynamic cooling systems.

### Control Systems Issues

The present technique is essentially a control system that employs reactive as well as proactive actuations to meet the goal of improving the overall energy efficiency of a data center. As such, it has to be scalable, stable and provide appropriate sense-and-actuate latencies. Another important requirement of the system is that the various control elements should act in a synchronized and coordinated manner, avoiding “power struggles” [RRT+08], where one control loop fights against another inadvertently.

On the control elements at the computing side, these control system requirements are met by a using a hierarchical implementation that uses independent control elements at each level and by using a progressive top-down approach to dictate the energy/performance goals of one level to be explicitly dictated by the control system at the immediately upper level. The hierarchical control mechanisms of the activities within a computing system also ensures its scalability:
separate control loops are used to ensure the energy budgets at the rack level and at the level of individual servers within the rack are monitored and managed separately. For large data centers, another level can be added to make the system more scalable, based on the allocation and control of the energy budgets for a set of neighboring racks.

The control of the computing equipment is based on the notion of update intervals within a sampling period, with sensor and model outputs collected at the end of each update period. At the end of a sampling period, the values of respective sensor and model data output are averaged, and control decisions taken at the end of a sampling period based on these average values, as introduced in [PKG 01]. This approach smooths out the impact of burst activities that are inevitable within a sampling interval and enables a stable control system for the computing elements.

### Hardware Overview

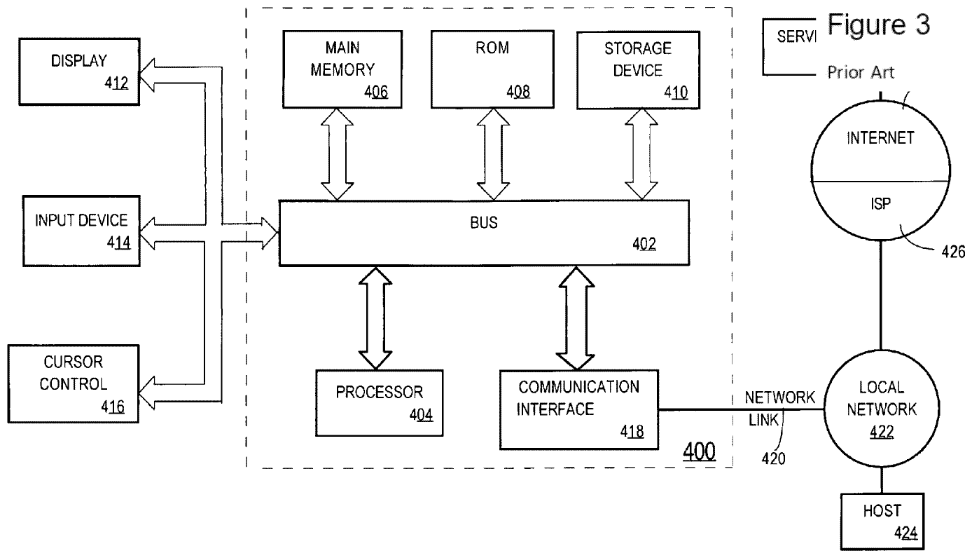

FIG. 3 (see U.S. Pat. No. 7,702,660, issued to Chan, expressly incorporated herein by reference), shows a block diagram that illustrates a computer system 400 upon which an embodiment of the invention may be implemented. Computer system 400 includes a bus 402 or other communication mechanism for communicating information, and a processor 404 coupled with bus 402 for processing information. Computer system 400 also includes a main memory 406, such as a random-access memory (RAM) or other dynamic storage device, coupled to bus 402 for storing information and instructions to be executed by processor 404. Main memory 406 also may be used for storing temporary variables or other intermediate information during execution of instructions to be executed by processor 404. Computer system 400 further includes a read only memory (ROM) 408 or other static storage device coupled to bus 402 for storing static information and instructions for processor 404. A storage device 410, such as a magnetic disk or optical disk, is provided and coupled to bus 402 for storing information and instructions.

Computer system 400 may be coupled via bus 402 to a display 412, such as a cathode ray tube (CRT) or liquid crystal flat panel display, for displaying information to a computer user. An input device 414, including alphanumeric and other keys, is coupled to bus 402 for communicating information and command selections to processor 404. Another type of user input device is cursor control 416, such as a mouse, a trackball, or cursor direction keys for communicating direction information and command selections to processor 404 and for controlling cursor movement on display 412. This input device typically has two degrees of freedom in two axes, a first axis (e.g., x) and a second axis (e.g., y), that allows the device to specify positions in a plane.

The invention is related to the use of computer system 400 for implementing the techniques described herein. According to one embodiment of the invention, those techniques are performed by computer system 400 in response to processor 404 executing one or more sequences of one or more instructions contained in main memory 406. Such instructions may be read into main memory 406 from another machine-readable medium, such as storage device 410. Execution of the sequences of instructions contained in main memory 406 causes processor 404 to perform the process steps described herein. In alternative embodiments, hard-wired circuitry may be used in place of or in combination with software instructions to implement the invention. Thus, embodiments of the invention are not limited to any specific combination of hardware circuitry and software.

The term “machine-readable medium” as used herein refers to any medium that participates in providing data that causes a machine to operate in a specific fashion. In an embodiment implemented using computer system 400, various machine-readable media are involved, for example, in providing instructions to processor 404 for execution. Such a medium may take many forms, including but not limited to, non-volatile media, volatile media, and transmission media. Non-volatile media includes, for example, optical or magnetic disks, such as storage device 410. Volatile media includes dynamic memory, such as main memory 406. Transmission media includes coaxial cables, copper wire and fiber optics, including the wires that comprise bus 402. Transmission media can also take the form of acoustic or light waves, such as those generated during radio-wave and infra-red data communications. All such media must be tangible to enable the instructions carried by the media to be detected by a physical mechanism that reads the instructions into a machine.

Common forms of machine-readable media include, for example, a floppy disk, a flexible disk, hard disk, magnetic tape, or any other magnetic medium, a CD-ROM, any other optical medium, punch cards, paper tape, any other physical medium with patterns of holes, a RAM, a PROM, and EPROM, a FLASH-EPROM, any other memory chip or cartridge, a carrier wave as described hereinafter, or any other medium from which a computer can read.

Various forms of machine-readable media may be involved in carrying one or more sequences of one or more instructions to processor 404 for execution. For example, the instructions may initially be carried on a magnetic disk of a remote computer. The remote computer can load the instructions into its dynamic memory and send the instructions over a telephone line using a modem. A modem local to computer system 400 can receive the data on the telephone line and use an infra-red transmitter to convert the data to an infra-red signal. An infra-red detector can receive the data carried in the infra-red signal and appropriate circuitry can place the data on bus 402. Bus 402 carries the data to main memory 406, from which processor 404 retrieves and executes the instructions. The instructions received by main memory 406 may optionally be stored on storage device 410 either before or after execution by processor 404.

Computer system 400 also includes a communication interface 418 coupled to bus 402. Communication interface 418 provides a two-way data communication coupling to a network link 420 that is connected to a local network 422. For example, communication interface 418 may be an integrated services digital network (ISDN) card or a modem to provide a data communication connection to a corresponding type of telephone line. As another example, communication interface 418 may be a local area network (LAN) card to provide a data communication connection to a compatible LAN. Wireless links may also be implemented. In any such implementation, communication interface 418 sends and receives electrical, electromagnetic or optical signals that carry digital data streams representing various types of information.

Network link 420 typically provides data communication through one or more networks to other data devices. For example, network link 420 may provide a connection through local network 422 to a host computer 424 or to data equipment operated by an Internet Service Provider (ISP) 426. ISP 426 in turn provides data communication services through the world wide packet data communication network now commonly referred to as the “Internet” 428. Local network 422 and Internet 428 both use electrical, electromagnetic or optical signals that carry digital data streams. The signals through the various networks and the signals on network link 420 and through communication interface 418, which carry the digital data to and from computer system 400, are exemplary forms of carrier waves transporting the information.

Computer system 400 can send messages and receive data, including program code, through the network(s), network link 420 and communication interface 418. In the Internet example, a server 430 might transmit a requested code for an application program through Internet 428, ISP 426, local network 422 and communication interface 418.

The received code may be executed by processor 404 as it is received, and/or stored in storage device 410, or other non-volatile storage for later execution. In this manner, computer system 400 may obtain application code in the form of a carrier wave.

In this description, several preferred embodiments were discussed. Persons skilled in the art will, undoubtedly, have other ideas as to how the systems and methods described herein may be used. It is understood that this broad invention is not limited to the embodiments discussed herein. Rather, the invention is limited only by the following claims.

### REFERENCES (EACH OF WHICH IS EXPRESSLY INCORPORATED BY REFERENCE)

U.S. Pat. No. 7,228,441 B 2
[BH 07] Luiz André Barroso and Urs Hölzle, “The Case for Energy-Proportional Computing”, IEEE Computer Magazine, December 2007.
[BH 09] Luiz André Barroso and Urs Hölzle, “The Datacenter as a Computer: An Introduction to the Design of Warehouse-Scale Machines”, Morgan-Claypool Publishers, 2009 (ISBN No. 9781598295566).
[ERK+08] D. Economou Suzanne Rivoire, Christos Kozyrakis, and Parthasarathy Ranganathan, “Full-system Power Analysis and Modeling for Server Environments”, in Proc. Workshop on Modeling Benchmarking and Simulation (MOBS) at the Int'l. Symposium on Computer Architecture, Boston, MA, June 2006.
[IBM 08a] IBM Corporation, IBM Tivoli Usage Accounting Manager V7.1 Handbook, IBM Redbook, March 2008.
[IBM 08b] IBM Corporation, Value Proposition for IBM Systems Director: Challenges of Operational Management for Enterprise Server Installations, IBM ITG Group, Management Brief (34 pages), November 2008.
[Ko 07] Jonathan G. Koomey, “Estimating Total Power Consumption By Servers in the U.S. and the World”, Analytics Press. February 2007. Also available at: enterprise.amd.com/us-en/AMD-Business/Technology-Home/Power-Management.aspx.
[LGT 08] Adam Lewis, Soumik Ghosh and N. -F. Tzeng, “Run-time Energy Consumption Estimation Based on Workload in Server Systems”, in Proc. of the HotPower 08 workshop, held in conjunction with the 2008 Usenix OSDI Symposium.
[LRC+08] Kevin Lim, Parthasarathy Ranganathan, Jichuan Chang, Chandrakant Patel, Trevor Mudge, Steven Reinhardt, “Understanding and Designing New Server Architectures for Emerging Warehouse-Computing Environments”, in Proc. of the 35th International Symposium on Computer Architecture, 2008, pp. 315-326.
[NSSJ 09] Ripal Nathuji, Ankit Somani, Karsten Schwan, and Yogendra Joshi, “CoolIT: Coordinating Facility and IT Management for Efficient Datacenters”, in Proc. of the HotPower 08 workshop, held in conjunction with the 2008 Usenix OSDI Symposium.
[PKG 01] Dmitry Ponomarev, Gurhan Kucuk and Kanad Ghose, “Reducing Power Requirements of Instruction Scheduling Through Dynamic Allocation of Multiple Datapath Resources”, in Proc. 34th IEEE/ACM International Symposium on Microarchitecture (MICRO-34), December 2001, pp. 90-101.
[RRT+08] Ramya Raghavendra, Parthasarathy Ranganathan, Vanish Talwar, Zhikui Wnag, and Xiaoyun Zhu, “No Power Struggles: Coordinated Multilevel Power Management for the Data Center”, in Proc. ACM Symposium on Architectural Support for Programming Languages and Operating Systems (ASPLOS), 2008.
[Sh 09] Stephen Shankland, “Google Uncloaks Once-Secret Server”, CNET News, Business Tech, April, 2009, available at: news.cnet.com/8301-1001_3-10209580-92.html.

[SBP+05] Ratnesh K. Sharma, Cullen Bash, Chandrakant D. Patel, Richard J. Friedrich, Jeffrey S. Chase: Balance of Power: Dynamic Thermal Management for Internet Data Centers. IEEE Internet Computing Vol. 9, No. 1, pp. 42-49, 2005.
[TGV 08] Qinghui Tang, Member, Sandeep K. S. Gupta, and Georgios Varsamopoulos, “Energy-Efficient, Thermal-Aware Task Scheduling for Homogeneous, High Performance Computing Data Centers: A Cyber-Physical Approach”, in IEEE Trans. On Parallel and Distributed Systems, November 2008 (vol. 19 no. 11) pp. 1458-1472.

What is claimed is:

1. A system for managing a data center having a plurality of servers, the servers having a stored queue of tasks prior to execution by the plurality of servers, comprising:
a plurality of sensors configured to monitor temperatures of a plurality of servers within the data center;
a processor configured to predict thermal trends for the plurality of servers, using a predictive model based on an empirical model of energy dissipation by the respective server as a function of the stored queue of tasks prior to execution by the plurality of servers, actual hardware activity counts of tasks executed by the plurality of servers, and the monitored temperatures; and
a cooling system management module configured to control a cooling system parameter in dependence on the thermal trend, to maintain a predetermined quality of service and operation of the plurality of servers within a predetermined thermal envelope and a control signal from the processor.

2. The system of claim 1, wherein the predictive model is adaptive and modified in dependence on actual outcomes determined by the plurality of sensors.

3. The system of claim 2, wherein the processor implements a genetic algorithm or self-tuning algorithm to search for a more efficient operating point during operation.

4. The system of claim 1, wherein the cooling system parameter comprises an inlet temperature or an air flow speed.

5. The system of claim 1, wherein the empirical model of energy dissipation is derived from software access to registers and counters which monitor operation of a semiconductor device within the plurality of servers.

6. The system of claim 1, wherein the cooling system management module treats energy expended in the cooling system as a first class resource explicitly scheduled to maximize overall energy-efficiency.

7. The system of claim 1, wherein the processor is further configured to redirect additional cooling to a region of the data center based on the predicted thermal trend to provide dynamic, directed cooling.

8. A computing system comprising:
a semiconductor device having a processor;
a set of registers and counters configured to monitor operation of the semiconductor device and track various types of activities;
an operating system configured to provide software access to the registers and counters;
an empirical energy dissipation model configured to determine energy consumed by the computing system as a function of measured values of actual activity counts read from the registers and counters and processes executed by the computing system; and
a power management actuator, configured to be controlled by the operating system in a proactive fashion in dependence on at least one unexecuted process, to permit the computing system to stay within an assigned energy consumption budget during subsequent execution of the unexecuted process, based on the determined energy consumed.

9. The computing system of claim 8, wherein the empirical energy dissipation model calculates the energy consumed within a sampling interval to guide local scheduling of processes for execution on the semiconductor device.

10. The computing system of claim 8, wherein the power management actuator dynamically changes a power-performance setting for the semiconductor device.

11. The computing system of claim 8, wherein the registers and counters include hardware instrumentation counters maintained in the processor.

12. The computing system of claim 8, wherein the assigned energy consumption budget is allocated by a global scheduler as a function of workload and actual server utilizations.

13. The computing system of claim 8, wherein the operating system is configured to smooth out an impact of burst activities within a sampling interval.

14. The computing system of claim 8, wherein the empirical energy dissipation model is executed on the semiconductor device as an additional process within a multitasking processing stream.

15. A method for proactively managing cooling in a data center, comprising:
collecting, at a sampling interval, a plurality of sensor measurements including temperature and airflow from a plurality of sensors distributed across a plurality of racks in the data center;
collecting, at the sampling interval, activity data representing a current computing load jobs executing on servers within the plurality of racks;
inputting the collected sensor measurements and the activity data into a thermal model, wherein the thermal model configured to predict future thermal conditions and energy efficiency of the data center;
predicting, using the thermal model, a thermal trend for at least rack based on the collected inputs;
controlling a cooling system having a cooling system parameter, for the plurality of racks, to minimize cooling energy consumption while maintaining the plurality of racks within a thermal envelope and a quality of service; and
automatically adjusting the cooling system parameter in dependence on a job within a queue of jobs, prior to execution of the job.

16. The method of claim 15, further comprising:
reading statistics maintained in hardware of the servers, and an operating system;
classifying jobs or phases of jobs into a plurality of categories using the statistics, the categories including at least a compute bound category and a memory bound category; and
learning energy-performance characteristics of the jobs or the phases of jobs as they execute, to update the thermal model.

17. The method of claim 16, further comprising allocating a job to a respective server based on a classification of the job and a classification of a preceding job executing on the respective server.

18. The method of claim 15, further comprising allocating complementary jobs to a single set of resources for concurrent execution, wherein the complementary tasks incur loads on different components of the computing system.

19. The method of claim 15, further comprising activating or deactivating servers or virtual machines (VMs) dependent on the queue of jobs, taking into account a latency and an energy overhead of activating or deactivating servers or virtual machines (VMs), while minimizing cooling energy consumption and maintaining the plurality of racks within the thermal envelope and the quality of service.

20. The method of claim 15, further comprising migrating a virtual machine (VM) dependent on the queue of jobs, taking into account an energy cost of migrating the virtual machine (VM), while minimizing cooling energy consumption and maintaining the plurality of racks within the thermal envelope and the quality of service.
